(19) United States
(12) Patent Application Publication
(10) Pub. No.: US 2011/0150812 A1
(43) Pub. Date: Jun. 23, 2011

(54) NATURAL CONDITIONING COSMETIC COMPOSITIONS

(75) Inventor: Jaimie MECCA, Clifton, NJ (US)

(73) Assignee: L'OREAL S.A., Paris (FR)

(21) Appl. No.: 12/949,087

(22) Filed: Nov. 18, 2010

Related U.S. Application Data
(60) Provisional application No. 61/288,904, filed on Dec. 22, 2009.

(51) Int. Cl. A61K 8/73; A61Q 5/12

(52) U.S. Cl. 424/70.19

(57) ABSTRACT

A composition and method of enhancing the appearance of a keratinous material involving the steps of: (a) providing a composition containing: (a) at least one colloidal agent comprising a combination of: (A) at least one microcrystalline cellulose compound and (B) at least one hydrocolloid compound, and wherein (A) is present in an amount greater than (B); (b) at least one polysaccharide; (c) at least one polyol ester; (d) optionally, at least one viscosity-modifying hydrocolloid; and (e) optionally, at least one emulsifier; and (f) a cosmetically acceptable carrier.

### REFERENCE TO PRIOR APPLICATION

This application claims the benefit of priority under 35 U.S.C. §119(e) from U.S. provisional application Ser. No. 61/288,904, filed Dec. 22, 2009, incorporated herein by reference in its entirety.

### BACKGROUND OF THE INVENTION

With many issues arising regarding products which may be harmful to the environment or chemically potent, a vast number of industries have been adopting a more “environmentally friendly” or “green” approach. From automotive, to fashion, to the food, cosmetic and pharmaceutical industries, companies have been experimenting with ways to create more environmentally safe products using environmentally friendly ingredients. In the cosmetic and personal care industry, such ingredients may also be naturally derived or natural-based ingredients that impart excellent cosmetic and aesthetic properties to the products.

At the same time, consumers of cosmetic and personal care products are now more conscious of the biological and environmental impacts of the products that they use, and therefore search for alternative products which utilize more environmentally friendly ingredients that are naturally derived. More particularly, consumers prefer those alternative products that are less harsh or irritating, while imparting desirable cosmetic effects, to the hair and to skin.

In contrast, conventional cosmetic compositions often contain ingredients such as cationically charged ingredients, ethoxylated materials and silicones. These materials, although known to provide good cosmetic benefits to hair and skin, may be less environmentally friendly and/or are found to be harsh or irritating to the skin and/or hair. Thus there is a need for alternative products that employ environmentally friendly ingredients and/or natural-based or naturally-derived ingredients, while at the same time, overcoming the formulation and manufacturing problems of making such products. More particularly, consumers seek such alternative hair cosmetic products that have the ability to effectively style and/or condition the hair, while providing a lustrous, healthy looking finish.

Thus, it has been surprisingly discovered that a cosmetic composition comprising a colloidal agent, a polysaccharide, and a polyol ester, wherein the composition does not require the use of silicones, cationically-charged ingredients, and ethoxylated materials, can provide a “natural” alternative to conventional hair cosmetic products. It has also been surprisingly and unexpectedly discovered that such a composition can be delivered to the hair in a sprayable form that remains stable and provides combability, manageability, moisture-retentivity, volume, shine, and softness to hair.

### BRIEF SUMMARY OF THE INVENTION

The present invention is directed to a cosmetic composition comprising:
(a) at least one colloidal agent comprising a combination of: (A) at least one microcrystalline cellulose compound and (B) at least one hydrocolloid compound, and wherein (A) is present in an amount greater than (B);
(b) at least one polysaccharide;
(c) at least one polyol ester;
(d) a cosmetically acceptable carrier;
(e) optionally, at least one viscosity-modifying hydrocolloid; and
(f) optionally, at least one emulsifier.

The present invention is also directed to a method of enhancing the appearance of a keratinous material involving the steps of:
(a) providing a composition containing:
   i. at least one colloidal agent comprising a combination of: (A) at least one microcrystalline cellulose compound and (B) at least one hydrocolloid compound, and wherein (A) is present in an amount greater than (B);
   ii. at least one polysaccharide;
   iii. at least one polyol ester;
   iv. a cosmetically acceptable carrier;
   v. optionally, at least one viscosity-modifying hydrocolloid;
   vi. optionally, at least one emulsifier; and
(b) applying said composition onto the keratinous material.

According to a preferred embodiment, the inventive compositions do not require the use of silicones, cationically-charged ingredients, and ethoxylated compounds in order to formulate environmentally-friendly products which impart shine, conditioning, and smoothing properties onto keratinous substrates treated therewith.

### DETAILED DESCRIPTION

Other than in the operating examples, or where otherwise indicated, all numbers expressing quantities of ingredients and/or ratios of ingredients are to be understood as being modified in all instances by the term “about”.

The term “hair styling” as used herein refers to styling or fixing hair into a desired configuration, such as imparting a style or temporary curl or set (straight or curly) to human hair and retaining or maintaining (grooming, restyling) a desired set or curl configuration.

The term “hair styling composition”, encompasses products that are applied to wet or dry or semi-dry hair before, during or after configuring the hair into the shape (curly or straight) desired, without limitation as to product form.

The terms hair “styling” and hair “fixative” agents as used herein, refer collectively to hair styling agents that are hair fixatives and film formers and which are topically applied to the hair to actively contribute to the ease of styling and/or holding of a section of hair, and to maintain the reshapability or smoothness of the hair or section of hair. Therefore, the present invention on hair styling compositions can include hair styling, hair fixative, and hair grooming products that conventionally are applied to wet or dry or semi-dry hair in the form of waxes, gels, rinses, sprays, emulsions (oil-in-water, water-in-oil or multiphase), such as lotions and creams, pomades, mousses, foams, shampoos, sprays, solids, such as sticks, semisolids and the like, or are applied from a hair styling aid having the hair styling composition impregnated therein or coated thereon, to leave the hair styling agent in contact with the hair for some period until removed, as by washing.

The phrase “does not irritate” or “less irritating” as used herein means that a composition employed in the methods of the invention, and such methods, does not result in a significant amount of detectable irritation (redness, burning, itching, adverse change) on the skin or scalp surface.

The term “colloidal agent” refers to a system in which finely divided coated or uncoated particles are dispersed within a continuous medium. The resulting dispersion may be stabilized using a dispersant or, where appropriate, without a dispersant. These particles may be of any form, for example, spherical, or elongated such as fibers.

The term “hydrocolloid” should be understood to mean a water-soluble macromolecule that does not alter the value of the water activity of the composition containing it.

The term “emulsifier” as used herein means any substance or agent that aids in the formation of an emulsion.

The term “emulsion” as used herein means a generally stable and homogeneous mixture of two liquids that do not normally mix (i.e., they are immiscible between themselves), such as vegetable oil and water. Emulsions can be true colloids or less stable mixtures, which tend to separate in a short time. An emulsion can often be broken down (i.e. the liquids separated) by factors such as mechanical manipulation, chemical effects and/or time.

The term “conditioning” or “condition” as used herein means imparting to at least one keratinous fiber at least one property chosen from combability, manageability, moisture-retentivity, luster, shine, and softness. The state of conditioning is evaluated by measuring, and comparing, the ease of combability of the treated hair and of the untreated hair in terms of combing work (gm-in).

“At least one” as used herein means one or more and thus includes individual components as well as mixtures/combinations.

It is to be understood that both the foregoing general description and the following detailed description are exemplary and explanatory only, and are not restrictive of the invention.

Colloidal Agent

The colloidal agent of the present invention comprises a combination of at least one microcrystalline cellulose compound and at least one hydrocolloid compound.

The at least one microcrystalline cellulose compound is present in the colloidal agent of the present invention in an amount greater than that of the hydrocolloid compound. More particularly, the microcrystalline cellulose compound is present in an amount of greater than 50% by weight, such as greater than 60% by weight, greater than 70% by weight, and greater than 80% by weight, all weights based on the total weight of the colloidal agent.

The at least one hydrocolloid compound includes soluble gums and salts and may be chosen from alginates (also known as algin), agar, carageenan, carboxymethylcellulose (cellulose gum), gum acacia, gum arabic, gum ghatti, gum karaya, gum tragacanth, guar gum, guar hydroxypropyltrimonium chloride, xanthan gum, gellan gum and mixtures thereof.

Preferred colloidal agents include, but are not limited to, Avicel PC611®, Avicel PC591®, Avicel CL 611 NF®, and Avicel PC815®, all commercially available from FMC Corp.

The at least one colloidal agent is present in the composition of the present invention at a level of from about 0.1% to about 10.0% by weight, preferably at a level of from about 0.5% to about 5.0%, preferably at a level of from about 1.0% to about 4.0% by weight, based on the total weight of the composition.

Polysaccharide

The compositions of the present invention additionally contain at least one polysaccharide that is different from the hydrocolloid in the colloidal agent described above.

The polysaccharide or polysaccharide derivative of the present invention may include a homopolysaccharide and/or a heteropolysaccharide and/or oligosaccharides, of animal, plant, microbial, bacterial or synthetic origin, and mixtures thereof.

Examples of polysaccharides or of polysaccharide derivatives which are suitable for carrying out the invention are glucans, amylose, glycogen, dextrans, β-glucans, fructosans, fructans, inulin, levane, mannans, xylans, lignins, arabans, galactans, galacturonans, chitin, glucuronoxylanes, arabinoxylanes, xyloglucans, galactomannans, glucomannans, arabinogalactans, carrageenans, agars, glycosaminoglucans, gum arabics, gum tragacanths, ghatti gums, karaya gums or mixtures thereof.

The preferred polysaccharides of the present invention are fructans, more specifically, inulins. The preferred inulins of the present invention are nonionic inulins such as those that are commercially available under the tradenames Inutec® H25P and Inutec® H25 from the supplier, Beneo Orafti.

Other preferred polysaccharides which can be used according to the invention are guar gums.

According to the invention, chemically modified or non-modified nonionic guar gums can be used.

The non-modified nonionic guar gums are, for example, the products sold under the name Vidogum GH 175 by the company Unipectine and under the name Jaguar C by the company Meyhall.

The modified nonionic guar gums which can be used according to the invention are preferably modified with C₁-C₆ hydroxyalkyl groups.

Among the hydroxyalkyl groups, mention may be made, for example, of hydroxymethyl, hydroxyethyl, hydroxypropyl and hydroxybutyl groups.

These guar gums are well known in the state of the art and can be prepared, for example, by reacting corresponding alkene oxides, such as, for example, propylene oxides, with guar gum so as to obtain a guar gum modified with hydroxypropyl groups.

The degree of hydroxyalkylation, which corresponds to the number of alkylene oxide molecules consumed by the number of free hydroxy functions present on the guar gum, preferably ranges from 0.4 to 1.2.

Such nonionic guar gums optionally modified with hydroxyalkyl groups are sold, for example, under the tradenames Jaguar HP8, Jaguar HP60 and Jaguar HP120, Jaguar DC293 and Jaguar HP105 by the company Meyhall, or under the name Galactasol 4H4FD2 by the company Aqualon.

The at least one polysaccharide is present in the composition of the present invention at a level of from about 0.01% to about 2.5% by weight, preferably at a level of from about 0.5% to about 2.0%, preferably at a level of from about 1.0% to about 2.0% by weight, based on the total weight of the composition.

Polyol Ester

Polyol esters as used in the present invention are generally esters of polyols or glycols or polyhydric alcohols and fatty acids.

Non-limiting examples of the at least one polyol ester include propylene glycol dioctanoate, neopentyl glycol diheptanoate, diethylene glycol diisononanoate, neopentyl glycol dicaprate, neopentyl glycol dicaprylate/dicaprate, neopentyl glycol diethyl hexanoate, neopentyl glycol diheptanoate, neopentyl glycol diisononanoate, neopentyl glycol diisostearate, neopentyl glycol dilaurate, propanediol dicaprylate, propanediol diisostearate, propanediol dicaprylate/caprate, propanediol dibennzoate, propanediol didecanoate, and anediol dihexanoate.

Other non-limiting examples of the at least one polyol ester include propylene glycol isostearate, propylene glycol caprylate, propylene glycol behenate, propylene glycol cocoate, propylene glycol laurate, propylene glycol linolenate, propylene glycol linoleate, propylene glycol myristate, propylene glycol oleate, and propylene glycol stearate.

Other non-limiting examples of the at least one polyol ester include polyol esters having alkoxylated groups such as PEG-55 propylene glycol oleate, PEG-6 propylene glycol caprylate/caprate, PEG-8 propylene glycol cocoate, PEG-25 propylene glycol stearate, and PEG-120 propylene glycol stearate.

The at least one polyol ester may also be chosen from esters of pentaerythritol and fatty acid and esters of dipentaerythritol and fatty acid such as pentaerythrityl tetraisostearate.

One preferred polyol ester of the present invention is propanediol dicaprylate.

The at least one polyol ester is present in the composition of the present invention at a level of from about 0.01% to about 4.0% by weight, preferably at a level of from about 0.1% to about 2.0%, preferably at a level of from about 0.25% to about 1.0% by weight, based on the total weight of the composition.

Cosmetically Acceptable Carrier

The cosmetically acceptable carrier may comprise a solvent chosen from water, organic solvents and mixtures thereof. Suitable examples of organic solvents include C1-C4 lower alkanols, such as ethanol and isopropanol; polyols and polyol ethers such as 2-butoxyethanol, propylene glycol, propylene glycol monomethyl ether, diethylene glycol ethyl ether, and diethylene glycol methyl ether; aromatic alcohols such as benzyl alcohol and phenoxyethanol, and mixtures thereof. Other examples of organic solvents include C5-C10 alkane diols such as pentanediols and octandiols, and hydrocarbons such as isododecane. Examples of solvents for use in the present invention are hexylene glycol and dipropylene glycol, and mixtures thereof.

A preferred cosmetically acceptable carrier comprises water as the solvent.

Another preferred cosmetically acceptable carrier comprises at least one organic solvent. More preferably, the cosmetically acceptable carrier comprises at least one polar organic solvent.

Another preferred cosmetically acceptable carrier comprises water and at least one organic solvent.

Viscosity-Modifying Hydrocolloid

Depending on the form of the product being made, i.e., spray, gel, cream, etc., the compositions of the present invention can optionally contain at least one viscosity-modifying hydrocolloid.

Nonlimiting examples of viscosity-modifying hydrocolloids that may be used in the present invention are xanthan gum, carragheenans, agar, Danish agar, maltodextrin, galactomanan, tragacanth, gellan gum, carboxymethylcellulose and modified guar gums.

Other nonlimiting examples of viscosity-modifying hydrocolloids may be chosen from cellulose and its derivatives; seaweed extracts; seed extracts; plant exudates; microorganism exudates; fruit extracts; gelling agents of animal origin; synthetic water-soluble gelling polymers; amphiphilic polymers; silicon derivatives; and their mixtures.

In more detail, these viscosity-modifying hydrocolloids may be chosen from:

cellulose and its derivatives such as carboxymethylcellulose, hydroxypropylcellulose, methylcellulose, hydroxypropylmethylcellulose and hydroxyethylcellulose, as well as modified celluloses, especially those modified by grafting of the alkyl group;

seaweed extracts such as agar agar, carragheenans and alginates;

seed extracts such as carob gum, guar gum, and modified guar gums, especially those modified by grafting of the alkyl group;

plant exudates such as gum arabic, karaya gum, gum tragacanth and gatty gum;

microorganism exudates such as xanthan gum;

fruit extracts such as pectins;

gelling agents of animal origin, such as gelatin and caseinates;

synthetic water-soluble gelling polymers such as crosslinked polyacrylic acids, including those crosslinked via an alkyl chain, such as CARBOPOL and PEMULEN from the company Goodrich;

silicon derivatives such as synthetic hectorites like the products LAPONITE RD and RDS sold by the company Waverly and aluminium magnesium silicates like the product VEEGUM sold by the company Vanderbilt;

polymers such as POLYCARE® sold by the company Rhone-Poulenc under the reference PS-20 and PS-32.

One preferred viscosity-modifying hydrocolloid is xanthan gum.

The viscosity-modifying hydrocolloid may be present in the composition of the present invention at a level of from about 0.01% to about 5.0% by weight, preferably at a level of from about 0.02% to about 3.0%, preferably at a level of from about 0.05% to about 1.0% by weight, based on the total weight of the composition.

Emulsifier

The compositions of the present invention can optionally contain at least one emulsifier.

The emulsifiers which can be used in the present invention are preferably chosen from nonionic compounds composed, on the one hand, of a lipophilic residue chosen, for example, from C₆-C₃₀ alkyl or acyl functional groups and, on the other hand, of a hydrophilic residue chosen, for example, from polyol groups (glycerol, glycol, glucose) and polyol ethers. Their HLB (Hydrophilic Lipophilic Balance) balance preferably ranges from 9 to 18, more preferably from 9.5 to 11.5.

Suitable nonionic emulsifiers used in the present invention include the addition products of ethylene oxide with fatty alcohols having 6 to 30 carbon atoms or with partial esters of polyols having 3 to 16 carbon atoms 17 and of fatty acids having 14 to 22 carbon atoms, and their mixtures.

Thus, for example, suitable addition products of ethylene oxide with fatty alcohols include an emulsifier corresponding to the formula (III): R—(O—CH₂—CH₂)ₙ—OH (III) in which R represents a saturated or unsaturated and linear or branched hydrocarbonaceous residue having from 8 to 30 carbon atoms and n represents a number ranging from 8 to 50, preferably from 8 to 30. Suitable addition products of ethylene oxide with behenyl alcohol include, for example, Beheneth-9 (9 mol of ethylene oxide) and Beheneth-10 (10 mol of ethylene oxide); those obtained with cetyl alcohol or with stearyl alcohol or with their mixtures, in particular those comprising 12 to 20 mol of ethylene oxide, such as, for example, those obtained from the mixture of cetyl alcohol and of stearyl alcohol (cetearyl alcohol), such as Ceteareth-12, Ceteareth-13, Ceteareth-14, Ceteareth-15, Ceteareth-16, Ceteareth-17, Ceteareth-18 or Ceteareth-20; those obtained from cetyl alcohol, such as Ceteth-14, Ceteth-15, Ceteth-16 or Ceteth-20; those obtained from stearyl alcohol, such as Steareth-13, 18 Steareth-14, Steareth-15, Steareth-16 or Steareth-20, and the mixtures of these various emulsifiers.

The addition products of ethylene oxide with partial esters of polyols and of fatty acids can be obtained by ethoxylation of glycerol partial esters of fatty acids or of sorbitol mono- or diesters of fatty acids. Use may be made, for example, of the addition products of 4 to 20 mol of ethylene oxide with one or more glycerol partial esters. The term “glycerol partial esters” is understood to mean, for example, the mixtures of mono-, di- and triglycerides of C₁₀-C₂₀ fatty acids obtained by esterification of one mole of glycerol by 1 or 2 mol of a C₁₀-C₂₀ fatty acid.

Other examples of emulsifiers of the present invention can include C₁₆-C₂₂ fatty alcohols or esters of C₃-C₆ polyols with C₁₄-C₂₂ fatty acids or monoesters of glycerin and fatty acids such as glyceryl stearate, and their mixtures.

One preferred emulsifier for use in the present invention is glyceryl stearate.

The optionally at least one emulsifier is present in the composition of the present invention at a level of from about 0.01% to about 1.5% by weight, preferably at a level of from about 0.01% to about 1.0%, preferably at a level of from about 0.1% to about 0.5% by weight, based on the total weight of the composition.

Additional Ingredients

The compositions of the present invention may further comprise light esters such as isopropyl myristate, dicaprylyl carbonate, ethyl acetate, isopropyl palmitate, dioctyl succinate, capryl isostearate, isopropyl isostearate, octyl isononanoate, Dicapryl Ether, Isononyl Isononanoate, and mixtures thereof.

The compositions of the present invention may also further comprise natural and synthetic oils such as caprylic/Capric triglycerides.

The cosmetic compositions and methods of the present invention can comprise, consist of, or consist essentially of the essential elements and limitations of the invention described herein, as well as any additional or optional ingredients, components, or limitations described herein or any otherwise useful ingredient found in personal care compositions intended for application to keratinous materials.

In one embodiment of the present invention, there is provided a method for contacting a keratinous material such as hair involving the steps of providing a leave-in composition containing at least one microcrystalline cellulose compound and at least one hydrocolloid compound, at least one polysaccharide, at least one polyol ester, a cosmetically acceptable carrier, optionally, at least one viscosity-modifying hydrocolloid; and optionally, at least one emulsifier.

In another embodiment of the present invention, there is provided a method for styling a keratinous material such as hair involving the steps of providing a hair styling composition containing at least one microcrystalline cellulose compound and at least one hydrocolloid compound, at least one polysaccharide, at least one polyol ester, a cosmetically acceptable carrier, optionally, at least one viscosity-modifying hydrocolloid; and optionally, at least one emulsifier.

In one preferred embodiment of the present invention, there is provided a cosmetic composition comprising at least one microcrystalline cellulose compound and at least one hydrocolloid compound, at least one polysaccharide, at least one polyol ester, a cosmetically acceptable carrier, optionally, at least one viscosity-modifying hydrocolloid; and optionally, at least one emulsifier.

In another preferred embodiment of the present invention, there is provided a cosmetic composition comprising at least one microcrystalline cellulose compound and at least one hydrocolloid compound, at least one polysaccharide, at least one polyol ester, a cosmetically acceptable carrier.

In another preferred embodiment of the present invention, there is provided a sprayable cosmetic composition comprising at least one microcrystalline cellulose compound and at least one hydrocolloid compound, at least one polysaccharide, at least one polyol ester, a cosmetically acceptable carrier, optionally, at least one viscosity-modifying hydrocolloid; and optionally, at least one emulsifier. This sprayable cosmetic composition may be applied onto hair for the purpose of styling and/or conditioning the hair.

According to one embodiment of the present invention, there is provided a process for contacting keratinous materials, such as hair or skin, by applying the inventive composition onto the keratinous material. The precise amount of inventive composition to be applied onto the material will depend on the degree of treatment desired.

The present invention relates to compositions for application onto hair such as styling sprays, waxes, pomades, creams, lotions, mousses, hair masques, and styling gels. The inventive compositions may be utilized in leave-on conditioners, permanent waving compositions, hair care products, hair treatment products, and hair styling products. The present invention may also be utilized in other personal care compositions for application onto skin, nails, and lips such as body washes and deodorants, skin care, sun care, lip care, nail care and facial care. In addition, the compositions of the present invention may be used in make up products such as foundation, powders, mascara, and lipsticks.

The composition of the present invention may be in any form. For example, it may be a paste, a liquid, a gel, a lotion, a cream, a spray, an emulsion, or a solid. The composition of the invention may be transparent or clear, including for example, a composition without pigments. The composition can also be a molded composition or cast as a stick or a dish. The inventive composition in one preferred embodiment is a liquid emulsion which is sprayable.

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the total composition, unless otherwise specified.

### Example 1

### Conditioning Spray

Natural Conditioning spray

| CHEMICAL NAME | % |
|---|---|
| GLYCERIN | 3 |
| INULIN | 1.4550 |
| MICROCRYSTALLINE CELLULOSE | 1.2750 |
| SODIUM BENZOATE | 0.50 |
| PROPANEDIOL DICAPRYLATE | 0.50 |
| ISOPROPYL MYRISTATE | 0.50 |
| CAPRYLYL GLYCOL | 0.50 |
| DICAPRYLYL CARBONATE | 0.499750 |
| GLYCERYL STEARATE SE | 0.30 |
| CETEARYL ALCOHOL | 0.30 |
| CELLULOSE GUM | 0.2250 |
| CITRIC ACID | 0.10 |
| XANTHAN GUM | 0.10 |
| WATER | Q.S. to 100 |

### Example 2

### Conditioning Spray

Natural Conditioning spray

| CHEMICAL NAME | % |
|---|---|
| GLYCERIN | 3 |
| INULIN | 1.4550 |
| MICROCRYSTALLINE CELLULOSE | 0.85 |
| SODIUM BENZOATE | 0.50 |
| PROPANEDIOL DICAPRYLATE | 0.50 |
| ISOPROPYL MYRISTATE | 0.50 |
| CAPRYLYL GLYCOL | 0.50 |
| DICAPRYLYL CARBONATE | 0.499750 |
| GLYCERYL STEARATE SE | 0.30 |
| CETEARYL ALCOHOL | 0.30 |
| CELLULOSE GUM | 0.15 |
| CITRIC ACID | 0.10 |
| XANTHAN GUM | 0.10 |
| WATER | Q.S. to 100 |

### Example 3

### Conditioning Cream

Natural Conditioning Cream

| CHEMICAL NAME | % |
|---|---|
| GLYCERIN | 3.0 |
| INULIN | 1.50 |
| MICROCRYSTALLINE CELLULOSE | 2.975 |
| SODIUM BENZOATE | 0.50 |
| PROPANEDIOL DICAPRYLATE | 0.10 |
| ISOPROPYL MYRISTATE | 0.10 |
| CAPRYLYL GLYCOL | 0.50 |
| DICAPRYLYL CARBONATE | 0.10 |
| CELLULOSE GUM | 0.525 |
| CITRIC ACID | 0.10 |
| XANTHAN GUM | 0.10 |
| Water | Q.S. to 100 |

### Evaluation of the Properties of the Inventive Composition on Hair

An evaluation study was conducted on the hair of ten female panelists with medium to very long hair. The study comprised two visits (initial and final) to the Evaluation center where expert evaluators trained to analyze the effects of cosmetic products applied onto hair were present, as well as at least four at home applications by the panelists in between the visits to the Evaluation center. In the first visit to the Evaluation center, the expert evaluator noted the initial hair description and condition of the panelists' hair. The panelists then shampooed their hair with their usual shampoo and conditioner (if applicable), applied the inventive composition, then dried and styled their hair as they normally would. Questionnaires were then filled out by the panelists on the application and cosmetic performance of the inventive composition. Next, the panelists took the unlabeled inventive composition home and used it in place of their usual leave-in product. They were requested not to use any additional styling products throughout the duration of the study and were to note the date of each shampoo and formula application performed at home. Finally, the panelists returned to the Evaluation center at which point the expert noted the condition of their hair. The panelists applied the inventive composition under the same conditions as their initial visit. They were then interviewed about their likes and dislikes of the inventive composition and the inventive composition was evaluated on both damp hair and on dry hair by the expert.

The inventive composition was found to be easy to use by the panelists and provided very good conditioning properties, particularly in regard to good smoothness and softness without leaving any residue or weighing the hair down. Detangling and ease of styling were also rated very positive by the majority of the panelists and good volume, control and hold were seen. The expert confirmed above average styling and cosmetic properties with good lightness and definition

Overall the product showed good combability, the hair was pliable and easy to style with good control, and the hair showed more volume. The hair was very soft and conditioned and appeared smooth and shiny.

What is claimed is:

1. A cosmetic composition comprising:
(a) at least one colloidal agent comprising a combination of: (A) at least one microcrystalline cellulose compound and (B) at least one hydrocolloid compound, and wherein (A) is present in an amount greater than (B);
(b) at least one polysaccharide;
(c) at least one polyol ester;
(d) a cosmetically acceptable carrier;
(e) optionally, at least one viscosity-modifying hydrocolloid; and
(f) optionally, at least one emulsifier;

2. The cosmetic composition according to claim 1, wherein the at least one colloidal agent comprises microcrystalline cellulose and cellulose gum.

3. The cosmetic composition according to claim 1, wherein the at least one colloidal agent comprises microcrystalline cellulose and algin.

4. The cosmetic composition according to claim 1, wherein the at least one colloidal agent is present in an amount ranging from about 0.1% to about 10% by weight relative to the total weight of the composition.

5. The cosmetic composition according to claim 1, wherein the at least one polysaccharide is chosen from: sucrose, maltose, fructose, glucose, honey, dextrins, amylose, amylopectin, inulin, xylans, xylose and mixtures thereof.

6. The cosmetic composition according to claim 1, wherein the at least one polysaccharide is present in an amount ranging from about 0.01% to about 2.5% by weight relative to the total weight of the composition.

7. The cosmetic composition according to claim 1, wherein the at least one polyol ester is chosen from propylene glycol dioctanoate, neopentyl glycol diheptanoate, diethylene glycol diisononanoate, propanediol dicaprylate, pentaerythritol esters and mixtures thereof.

8. The cosmetic composition according to claim 1, wherein the at least one polyol ester is present in an amount ranging from about 0.1% to about 2% by weight relative to the total weight of the composition.

9. The cosmetic composition according to claim 1, wherein the cosmetically acceptable carrier is chosen from water, organic solvents and mixtures thereof.

10. The cosmetic composition according to claim 1, wherein the at least one viscosity-modifying hydrocolloid is chosen from alginates (also known as algin), carageenan, carboxymethylcellulose, gum acacia, gum arabic, gum ghatti, gum karaya, gum tragacanth, guar gum, guar hydroxypropyltrimonium chloride, xanthan gum, gellan gum and mixtures thereof.

11. The cosmetic composition according to claim 1, wherein the at least one viscosity-modifying hydrocolloid is present in an amount ranging from about 0.01% to about 1% by weight relative to the total weight of the composition.

12. The cosmetic composition according to claim 1, wherein the at least one emulsifier is chosen from glyceryl stearate.

13. The cosmetic composition according to claim 1, wherein the at least one emulsifier is present in an amount ranging from about 0.01% to about 1.5% by weight relative to the total weight of the composition.

14. The cosmetic composition according to claim 1, wherein the composition does not require the use of silicones, cationically-charged ingredients, and ethoxylated compounds.

15. A method of enhancing the appearance of a keratinous material involving the steps of:
(a) providing a composition containing:
i. at least one colloidal agent comprising a combination of: (A) at least one microcrystalline cellulose compound and (B) at least one hydrocolloid compound, and wherein (A) is present in an amount greater than (B);
ii. at least one polysaccharide;
iii. at least one polyol ester;
iv. a cosmetically acceptable carrier;
v. optionally, at least one viscosity-modifying hydrocolloid;
vi. optionally, at least one emulsifier; and
(b) applying said composition onto the keratinous material.

16. The method according to claim 15, wherein the at least one colloidal agent comprises microcrystalline cellulose and cellulose gum.

17. The method according to claim 15, wherein the composition does not require the use of silicones, cationically charged ingredients, and ethoxylated materials.

18. A sprayable cosmetic composition comprising:
(a) at least one colloidal agent comprising a combination of: (A) at least one microcrystalline cellulose compound and (B) at least one hydrocolloid compound, and wherein (A) is present in an amount greater than (B);
(b) at least one polysaccharide;
(c) at least one polyol ester;
(d) a cosmetically acceptable carrier;
(e) optionally, at least one viscosity-modifying hydrocolloid; and
(f) optionally, at least one emulsifier;

19. The sprayable cosmetic composition according to claim 18, wherein the at least one colloidal agent comprises microcrystalline cellulose and cellulose gum.

20. The sprayable cosmetic composition according to claim 18, wherein the sprayable cosmetic composition does not require the use of silicones, cationically charged ingredients, and ethoxylated materials.
